(19) United States
(12) Patent Application Publication
(10) Pub. No.: US 2024/0293819 A1
(43) Pub. Date: Sep. 5, 2024

(54) SAMPLE PROCESSING DEVICE

(71) Applicant: UNIQ BIOTECH LTD., Delta (CA)

(72) Inventors: Yunchuan (Morgan) ZHANG, Delta (CA); Danyang (Sandy) SHEN, Victoria (CA); Fernanda Alexandre do AMARAL, Delta (CA); Yisha (Serena) CHAI, Delta (CA); Liliana Jimenez SANCHEZ, Delta (CA)

(21) Appl. No.: 18/592,264

(22) Filed: Feb. 29, 2024

Related U.S. Application Data
(60) Provisional application No. 63/487,902, filed on Mar. 2, 2023.

(51) Int. Cl. B01L 3/00; G01N 33/543

(52) U.S. Cl. CPC B01L 3/508; G01N 33/543; B01L 2200/16; B01L 2300/042; B01L 2300/0681; B01L 2300/0832

(57) ABSTRACT

A sample testing device, a method of preparing a filter for use with the sample testing device, a kit comprising the sample testing device, and a process for detecting antibodies and/or antigens are disclosed.

### CROSS-REFERENCE TO RELATED APPLICATION

This application claims priority from U.S. application No. 63/487,902 filed 2 Mar. 2023 and entitled SAMPLE PROCESSING DEVICE which is hereby incorporated herein by reference for all purposes. For purposes of the United States of America, this application claims the benefit under 35 U.S.C. § 119 of U.S. application No. 63/487,902 filed 2 Mar. 2023 and entitled SAMPLE PROCESSING DEVICE which is hereby incorporated herein by reference for all purposes.

### FIELD OF THE INVENTION

The invention pertains to a sample processing device, particularly one for use in the detection of antibodies or antigens in a sample of a subject.

### BACKGROUND

Sample processing devices, in particular, those for use in the detection of antigens or antibodies in a sample, are known in the art. Designs of conventional sample processing devices are not easy to use, typically requiring the user to perform a series of processing steps before a result can be obtained. The present invention is directed to an improved sample processing device, in particular, one that is user-friendly and can also provide rapid and sensitive detection.

### SUMMARY

An aspect of the invention provides a sample processing device for the detection of an antibody or antigen in a sample. The sample processing device comprises a buffer container for receiving a buffer fluid therein, and a buffer cap snuggly mountable to an open end of the buffer container. The buffer cap has a fluid outlet end and an opposing fluid inlet end, and the fluid inlet end is contactable with the buffer container. The sample processing device also comprises a filter arranged at the fluid inlet end of the buffer cap. The filter may be coated with a color conjugate comprising a color reagent coupled to a polymer. In some embodiments, the filter and the color conjugate form a single unit. In some embodiments, the color reagent comprises colloidal gold nanoparticles.

An aspect of the invention provides a method of preparing a filter for use in the sample processing device. The method comprises preparing a solution comprising compounds containing gold ions, such as hydrogen tetrachloroaurate (HAuCl₄) and gold chloride (AuCl₃), and a reducing agent. A weight concentration of the hydrogen tetrachloroaurate and/or the gold chloride in the solution may be greater than 0.05%. The solution may be heated to a temperature in the range of from about 20° C. to about 100° C. to form a colloidal gold solution. A polymer may be added to the colloidal gold solution to form a colloidal gold conjugate solution. In some embodiments, the filter is prepared by soaking the filter into the colloidal gold conjugate solution. The colloidal gold conjugate coated filter may then be dried before incorporation into the buffer cap.

An aspect of the invention provides a kit for detection of target antigens and/or antibodies in a sample. The kit comprises the sample processing device, a test device comprising a membrane with a test region and a control region, and a buffer.

An aspect of the invention provides a process for detecting antibodies or antigens in a sample. The process may involve collecting the sample, mixing a buffer fluid such as a sample extraction fluid with the sample in the buffer container to form a mixture, and snuggly mounting a buffer cap to an open end of the buffer container. The mixture may then be allowed to pass through a filter coated with a color conjugate before flowing the mixture out of the buffer cap through a fluid outlet end, and allowing the mixture to flow through a test region of a membrane of the test device. In some embodiments, the test region of the membrane comprises a polymer immobilized thereon adapted to bind to a target antibody or antigen in the sample. In some embodiments, the mixture is caused to flow through a control region of the membrane. In some embodiments, the test region of the membrane comprises a polymer such as Protein A or Protein A like molecules immobilized thereon.

Further aspects of the invention and features of specific embodiments of the invention are described below.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a perspective view of a sample processing device according to an example embodiment of the invention.

FIG. 2 is a schematic diagram illustrating an example filter coated with a color conjugate of the FIG. 1 sample processing device.

FIG. 3 is a flow chart illustrating the steps of making a colloidal gold conjugate solution and preparing a filter coated with the colloidal gold conjugate solution according to an example embodiment of the invention.

FIG. 4 is a schematic diagram illustrating a detection kit comprising the FIG. 1 sample processing device according to an example embodiment of the invention.

FIG. 5 is an exploded view of an example test device included in the FIG. 4 detection kit.

FIG. 6A is a schematic diagram illustrating an example of polymer interactions at the test region of the membrane in the FIG. 5 test device in which detectable amounts of target antigens are present in the sample.

FIG. 6B is a schematic diagram illustrating an example of polymer interactions at the control region of the membrane in the FIG. 5 test device.

FIG. 7 is a flow chart illustrating the steps of processing a sample in the detection of antibodies and/or antigens according to an example embodiment of the invention.

FIG. 8A is a schematic diagram illustrating a second example of polymer interactions at the test region of the membrane in the FIG. 5 test device in which detectable amounts of target antibodies are present in the sample.

FIG. 8B is a schematic diagram illustrating a second example of polymer interactions at the control region of the membrane in the FIG. 5 test device.

### DETAILED DESCRIPTION

### Sample Processing Device

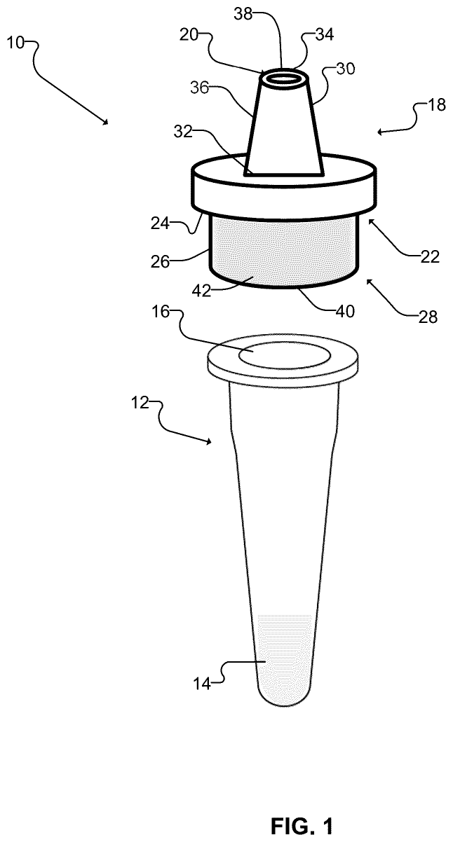

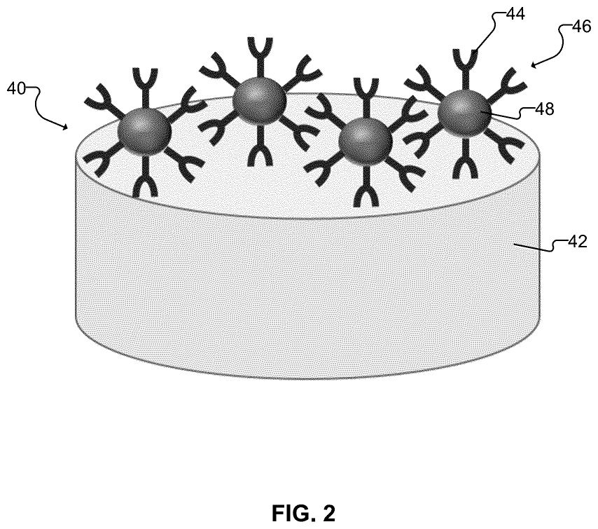

Referring to FIGS. 1 and 2, in one embodiment, the apparatus of the invention is a sample processing device 10. The sample processing device 10 comprises a buffer container 12 for receiving a buffer fluid 14 therein. The buffer container 12 has an open end 16 through which the buffer fluid 14 may be introduced. The device 10 has a buffer cap 18 snuggly mountable to the opening 16 of the buffer container 12. The mounting of the buffer cap 18 to the opening 16 closes the buffer container 12. In some embodiments, the buffer cap 18 is securely attached to the buffer container 12 such that the buffer cap 18 remains attached to the buffer container 12 when the buffer container 12 is opened, or in an open configuration. In some embodiments, the buffer cap 18 is detached from the buffer container 12 such that when the buffer container 12 is in the open configuration, the buffer cap 18 and buffer container 12 form separate units.

The buffer cap 18 has a fluid outlet end 20 and an opposing fluid inlet end 22. The fluid outlet end 20 and the fluid inlet end 22 may be in fluid communication with one another, such that fluid passes from the fluid inlet end 22 through to the fluid outlet end 20 to flow out of the buffer cap 18, and thereby out of the buffer container 12. The fluid inlet end 22 may be contactable with the buffer container 12. In some embodiments, the fluid inlet end 22 comprises a base 24 and a wall 26 projecting outwardly from the base 24. The wall 26 may define an opening 28. In some embodiments, the opening 28 has a cylindrical shape with a substantially circular cross-section. In some embodiments, the fluid outlet end comprises an elongated nozzle tip 30. The nozzle tip 30 may comprise a first end 32 secured to the base 24 of the fluid inlet end 22, an opposing second end 34, a side wall 36 extending between the first and second ends 32, 34, and a channel 38 defined by the side wall 36 through which the fluid flows through. In some embodiments, the cross-sectional area of the channel 38 decreases as it extends from the first end 32 to the first end 34 such that the cross-sectional area of the second end 34 is smaller than the cross-sectional area of the first end 32. The cross-sectional area of the second end 34 may be dimensioned to limit and/or control the fluid discharge from the buffer container 12.

A filter 40 may be arranged at the fluid inlet end 22 of the buffer cap 18. The filter is positioned such that fluid contacts and flows through the filter 40 as it flows out of the fluid outlet end 20 of the buffer cap 18. In some embodiments, the filter 40 is fitted into the buffer cap 18. In some embodiments, the filter 40 is snuggly fitted within the opening 28 of the buffer cap 18. In some embodiments, the filter 40 is disc and/or cylindrical shaped. The filter 40 may be porous such that the filter 40 may be adapted to block unwanted substances in the sample from passing therethrough. The pore size of the filter 40 may be selected depending on the sample being processed. In some example embodiments, the sample to be processed comprises fluid collected from a nasal swab. The pore size of the filter 40 selected for such application may for example be in the range of from about 0.45 microns to about 1.2 microns. Any suitable material may be used for the filter 40. Examples of suitable materials that may be used include paper, cellulose ester (for example including cellulose monoacetate, cellulose diacetate, cellulose triacetate, cellulose propionate, cellulose butyrate, cellulose acetobutyrate, cellulose nitrate, methyl cellulose, ethyl cellulose and mixtures thereof), triacetate fibers including for example, cellulose triacetate fibers, and mixtures thereof. In some embodiments, the filter 40 is a commercial product sold by companies such as EssentraComponents™, BMB Process Filters™, Nemuno Banga™, Bristol Myers Squibb (BMS)™, and Filtrona™. In some embodiments, the filter 40 has a diameter in the range from about 2.5 mm to about 7.5 mm. In some embodiments, the filter has a height in the range of from about 4 mm to about 20 mm.

The filter 40 may be coated with a color reagent 42. In some embodiments, the filter 40 may be impregnated with the color reagent 42. In some embodiments, a polymer 44 is bound to the color reagent 42 to form a color conjugate 46. The color conjugate 46 may comprise colloidal nanoparticles 48 such as colloidal gold nanoparticles and the polymer 44 conjugated to the nanoparticles 48. In some embodiments, the polymer 44 comprises a protein. In some embodiments, the polymer 44 comprises an antigen or antibody suitable for the detection of a target antigen or antibody in a sample. In some embodiments, the polymer 44 comprises a molecule such as Protein A or Protein-A like molecules. As used herein, “Protein A” is a native protein A or a functional derivative thereof. Protein A is a cell wall component produced by strains of Staphylococcus aureus. The polymer 44 may comprise other suitable molecules such as Protein L (i.e., a cell wall molecule of the bacterial species Peptostreptococcus magnus with affinity for immunoglobulin (Ig) light chains) such as Capto™ L resins, Protein G (i.e., an immunoglobulin (IgG)-binding bacterial cell wall protein isolated from group G streptococci), and Protein A/G.

In some embodiments, the filter 40 and the color conjugate 46 form a single unit. In such embodiments, the filter 40 functions both as a separator (i.e., to separate out unwanted substances from the sample) and as a color indicator (i.e., to provide color change as an indicator of a detectable amount of antigen or antibody present).

### Synthesis of Colloidal Gold Conjugate

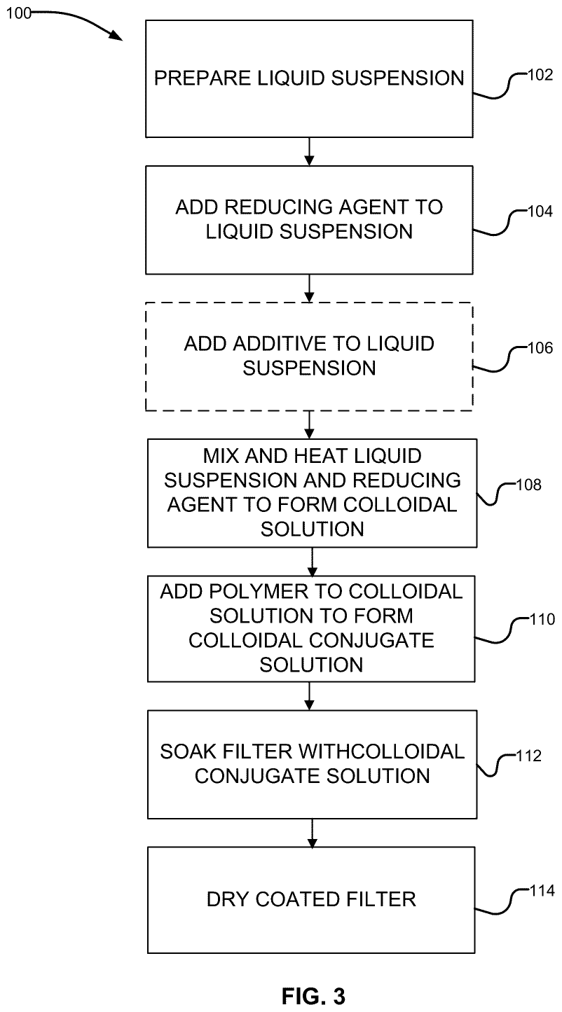

In some embodiments of the invention, the color conjugate 46 comprises colloidal nanoparticles 48 and a polymer 44. Aspects of the invention involve methods of synthesizing a colloidal conjugate such as a colloidal gold conjugate. Referring to FIG. 3, in some embodiments, the method 100 comprises preparing a liquid suspension by dissolving one or more compounds containing gold ions in a fluid such as water (block 102). In some embodiments, the one or more compounds contain gold and chlorine ions. In some example embodiments, the one or more compounds comprise hydrogen tetrachloroaurate (HAuCl₄). In some example embodiments, the one or more compounds comprise gold (III) chloride (AuCl₃), gold(I) chloride (AuCl), and potassium cyanate (K[Au(CN)₂]). A reducing agent may be added to the liquid suspension (block 104).

Any reducing agent suitable for reducing another substance, by donating one or more electrons to the another substance, may be used. In some embodiments, a reducing agent is an agent capable of reducing gold ions to gold atoms. Examples of suitable reducing agents include alcohols (e.g., methanol, ethanol, 1-propanol, ethylene glycol, 2-propanol and 2-butanol, glycerin), sugars (e.g., glucose, fructose, glyceraldehyde, lactose, arabinose, and maltose), aldehydes (e.g., formaldehyde and acetaldehyde), organic acids (e.g., citric acid, tannic acid, ascorbic acid), organic salts (e.g., sodium citrate, potassium citrate, ammonium citrate, sodium ascorbate, potassium ascorbate sodium borohydride and potassium borohydride) and inorganic salts (e.g., hydrazine hydrochloride, and hydrazine sulfate). In one example embodiment, the reducing agent comprises sodium citrate.

In some embodiments, one or more additives are optionally added to the liquid suspension before or after the addition of the reducing agent (block 106). In some embodiments, the one or more additives comprise a stabilizing agent. The stabilizer agent may for example be a compound capable of preventing the gold colloidal nanoparticles from aggregating. Examples of suitable compounds for use as stabilizing agents include but not limited to poly(ethylene glycol) (PEG), polypropylene glycol (PG), glycerol, poly(N-(2-hydroxypropyl) methacrylamide) (PHPMA), polyvinylpyrrolidone (PVP), and bovine serum albumin (BSA).

In some embodiments, a weight concentration of the one or more gold-ion containing compounds (e.g., hydrogen tetrachloroaurate and/or gold chloride) in the liquid suspension is greater than about 0.01%, or greater than about 0.05%, or greater than about 0.1%. In some embodiments, the weight concentration of the one or more gold-ion containing compounds (e.g., hydrogen tetrachloroaurate and/or gold chloride) in the liquid suspension is in the range of from about 0.1% to about 1.0%, or in the range of from about 0.1% to about 0.5%.

In some embodiments, the liquid suspension, the reducing agent, and the one or more additives (if present) are mixed and heated to a temperature to form a colloidal gold solution (block 108). In some embodiments, the temperature is in the range of from about 20° C. to about 100° C. In some embodiments, the temperature is in the range of from 45° C. to 55° C. In some embodiments, the liquid suspension, the reducing agent, and the one or more additives (if present) are heated for a time period in the range of from about 15 minutes to about 1 hour, and in some embodiments, from about 25 minutes to about 45 minutes.

A polymer may be added to the colloidal gold solution to form a colloidal gold conjugate solution (block 110). The polymer may for example be a protein such as an antigen or antibody suitable for the detection of a target antigen or antibody in a sample. This step may for example be performed at room temperature, for example at a temperature in the range of from about 15° C. to about 28° C.

In some embodiments, the colloidal gold conjugate solution has an optical density (OD) in the range of from about 10 to about 30 at a wavelength between about 500 nm and about 550 nm.

The colloidal conjugate need not be made from gold nanoparticles. The colloidal conjugate may for example be made from any one or more of silver nanoparticles, dye nanoparticles, and/or carbon nanoparticles. Suitable compounds containing the appropriate ions are selected for use in such embodiments to produce the desired colloidal conjugate. For example, in embodiments in which the colloidal nanoparticles 48 comprises colloidal silver nanoparticles, one or more compounds containing silver ions are selected. A suitable compound may for example be silver chloride.

Preparing the Filter with Colloidal Conjugate

In some embodiments, the filter 40 is coated and/or impregnated with the colloidal conjugate solution, such as the colloidal gold conjugate solution. The filter 40 may be coated and/or impregnated with the colloidal conjugate solution by soaking the filter 40 in the colloidal conjugate solution (block 112). In some embodiments, the filter 40 is soaked in the colloidal conjugate solution for a time period of from about 1 minutes to about 30 minutes, and in some embodiments, from about 5 minutes to about 15 minutes.

In some embodiments, the colloidal conjugate solution-coated filter 40 is dried to an acceptable moisture content (block 114). In some embodiments, the acceptable moisture content is lower than 5%, or in the range of from about 2.5% to about 5%. In some embodiments, the colloidal conjugate solution-coated filter 40 is dried by applying heat at a temperature for a time interval. The temperature may for example be in the range of from 25° C. to 50° C., or in some example embodiments, from about 35° C. to about 40° C. The time interval may for example be in the range of from about 10 to about 30 hours, or in some example embodiments, about 10 hours to about 20 hours.

### Antigen or Antibody Detection Kit

Aspects of the invention involve an antigen or antibody detection kit. The detection kit may comprise reagents and/or tools for detecting the presence of a target antigen or antibody. The detection kit may for example be used for the detection of infectious diseases such as the coronavirus, influenza virus, RSV, HIV/AIDS, dengue, syphilis, gonorrhea, chlamydia, herpes, hepatitis, etc.

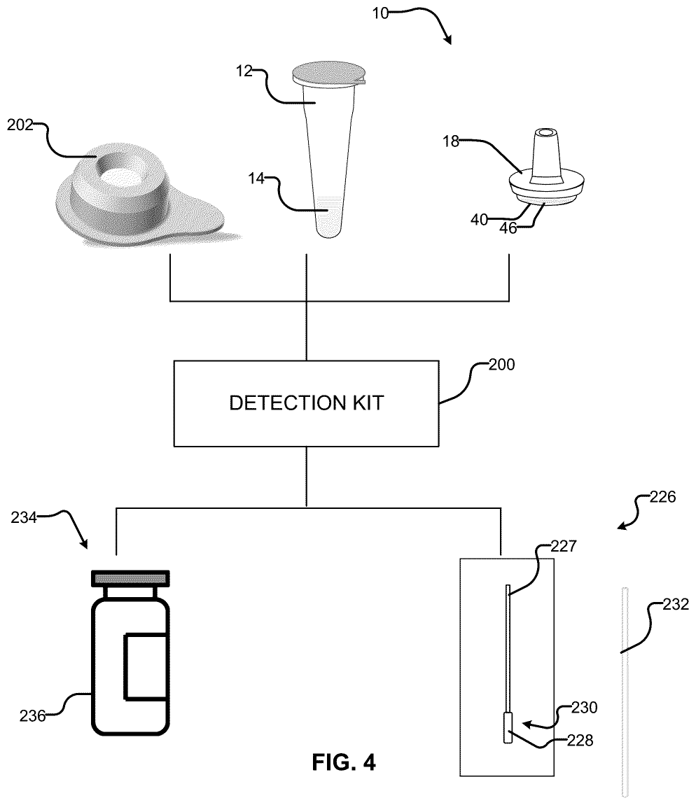

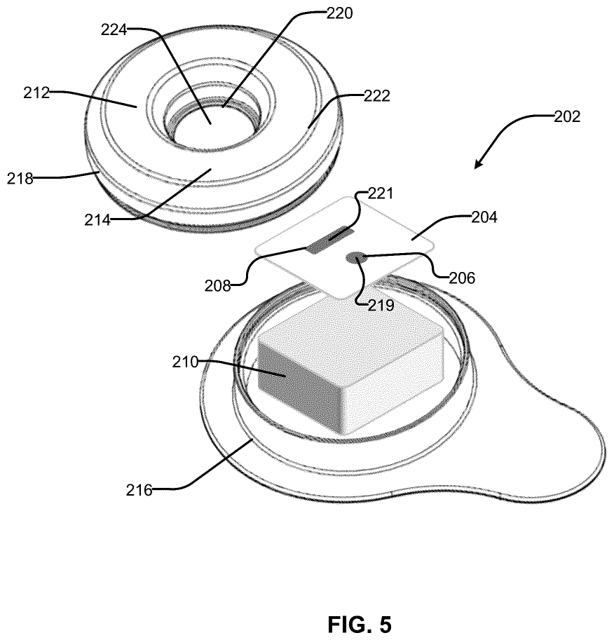

Referring to FIGS. 4 and 5, in some embodiments, the detection kit 200 comprises a test device 202, the sample processing device 10 comprising the buffer container 12, the buffer cap 18 and the filter 40 coated with the color conjugate 46. In some embodiments, the filter 40 coated with the color conjugate 46 is snuggly fitted within the buffer cap 18. In some embodiments, the detection kit 200 includes the buffer fluid 14 which may be received within the sample processing device 10. In some embodiments, the buffer fluid 14 comprises a sample extraction buffer. The sample extraction buffer may for example be used to inactivate the viruses or microbes in the sample to release viral or microbial antigens. Any sample extraction buffer suitable for the sample and/or virus and/or microbes and/or antigens may be used. In some example embodiments, the sample extraction buffer comprises one or more of borate buffer, PBS buffer, Tris buffer, including surfactants such as Tween-20 and Triton™ X-100, and sodium azide.

The test device 202 comprises a membrane 204 for example a nitrocellulose membrane. In some embodiments, the membrane 204 comprises at least a test region 206 and a control region 208.

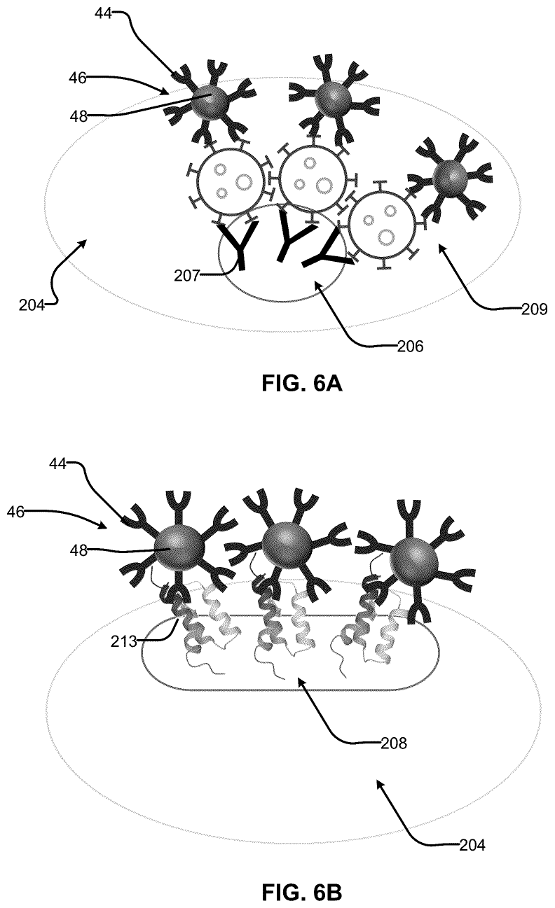

Referring to FIG. 6A, in some embodiments, the test region 206 comprises a molecule 207 immobilized thereon. In some embodiments, the molecule 207 is a protein such as an antibody or an antigen. The molecule may for example comprise an anti-protein antibody designed to capture the desired protein in the sample (e.g., the target viral antigen). In other embodiments, the protein comprises an antigen designed to capture the desired antibody in the sample.

Referring to FIG. 6B, in some embodiments, the control region 208 comprises a control polymer 213 such as a protein immobilized thereon. The control polymer may serve to capture the polymer (e.g., the antibody which targets against the target viral antigen) which is bound to the colloidal nanoparticles 48 of the colloidal conjugate 46. The polymer 213 may for example be Protein A or other Protein A-like molecules, Protein L (e.g., Capto™ L resins), Protein G, Protein A/G, and/or specific antibodies; however other molecules suitable for use as a control may be used to determine whether the diagnostic assay has been performed properly. The membrane 204 may include suitable indicator signs (e.g., lines, dots, etc.) at the test and control regions 206, 208 to display the test result.

In some embodiments, an absorbent pad 210 is positioned under the membrane 204 arranged to receive the fluid and/or absorb the fluid after it flows through the membrane 204. Any suitable material may be used to form the absorbent pad 210. Suitable materials include, for example, hydrophilic polyethylene materials or pads, glass fiber filter paper or pads, desiccated paper, paper pulp, fabric, and the like.

In some embodiments, a housing 212 is provided to receive the membrane 204 and the absorbent pad 210. In some embodiments, the housing 212 comprises an upper housing 214 and an opposing base 216 fitted to the upper housing 214. The upper housing 214 may comprise an outer edge 218 and an inner edge 220 radially spaced from the outer edge 218, a wall 222 extending from the outer and inner edge 218, 220, and an opening 224 defined by the outer and inner edges 218, 220. The opening 224 may be dimensioned to expose at least the indicator signs 219, 221 of the test and control regions 206, 208 of the membrane 204.

In some embodiments, the detection kit 200 comprises a sample collector 226 for collecting a biological sample. In some embodiments, the sample collector 226 comprises an elongated sample swab 227 having a swab tip 228 at a distal end 230 thereof. The sample collector 226 may be suitably dimensioned to collect a biological sample from a desired location in a subject, for example a human or animal. In the example embodiments, the elongated sample swab 227 is dimensioned to collect a nasal cavity and/or the throat of a subject. In other example embodiments, the sample collector 226 comprises a capillary sampling tube 232 adapted to collect a blood sample from a subject.

In some example embodiments, the kit 200 comprises one or more additional buffer reagents 234. The one or more additional buffer reagents 234 may be provided based on the sample to be processed. For example, in one embodiment, the one or more additional buffer reagents 234 comprises a wash buffer 236. The wash buffer 236 may for example be used to wash away excess stains, blood, proteins, etc. on the membrane 204 after the sample is caused to flow through the membrane 204.

Other suitable accessories and/or reagents may optionally be included in the kit 200. Such suitable accessories may for example include a safety lancet (for example for drawing a blood sample), alcohol pad, tube rack, etc.

### Process for Antibody or Antigen Detection

Aspects of the invention involve a method for detecting an antibody or antigen. In some example embodiments, the process comprises using the sample processing device and/or the kit 200 in the detection of the antibody or antigen.

Referring to FIGS. 6A, 6B, 7, 8A and 8B, in some embodiments, the process 300 begins with collecting a sample from a subject, such as a human or animal subject (block 301). The sample may for example be collected from the nasal cavity or the oral cavity. In another example, a blood sample from a subject may be collected. The sample may then be mixed with the buffer fluid 14 to form a mixture in the buffer container 12 of the sample processing device 10 (block 302). The sample may for example be a fluid sample collected from a human or animal subject. The buffer cap 18 may then be snuggly mounted to the open end 16 of the buffer container 12 (block 304). The mixture may be caused to flow out of the buffer cap 18 onto the membrane 204 of the test device 202 by first passing through the filter 40 which is impregnated with the color conjugate 46 (block 306). In some embodiments, the color conjugate 46 comprises the colloidal gold nanoparticles 48 and the polymer 44 conjugated to the nanoparticles 48. In some embodiments, the passing of the mixture through the filter 40 results in releasing the color conjugate 46. In some embodiments in which the sample comprises detectable amounts of target antigen or antibody, the passing of the mixture through the filter 40 results in binding of the polymer 44 with the target antigen or antibody in the sample to form a complex 209, and releasing the complex. The mixture comprising the color conjugate 46 and/or the complex 209 may be caused to flow onto the test region 206 of the membrane 204 (block 308). The test region 206 may for example comprise the polymer 207 immobilized thereon designed for capturing a target antibody or antigen from the sample.

The mixture comprising the color conjugate 46 and/or the complex 209 may be caused to flow onto the control region 208 of the membrane 204 (block 310). The control region 208 may for example comprise the control polymer 213 such as Protein A or Protein A like molecules immobilized thereon designed for capturing the polymer 44 conjugated to the nanoparticles 48 of the color conjugate 46. A color indicator, indicating the results, may be displayed on the control region 208 and the test region 206 (if a detectable amount of target antigens or antibodies is present in the sample).

In some embodiments, a wash buffer is caused to flow onto the membrane 204, or in some embodiments, the test region 206 and/or the control region 208 (block 312). The flowing through of the wash buffer onto the membrane 204 may facilitate washing away excess stains, blood, proteins, etc. on the membrane 204 after the sample is caused to flow through the membrane 204. In some embodiments, the step of flowing the wash buffer onto the membrane is performed only if certain samples are being tested, such as blood samples.

A Limit of Detection (LoD) in the range of from about 0.5×10³ TCID₅₀/mL to about 5×10³ TCID₅₀/mL may be achieved in the detection of antigens in a sample using the methods and/or detection kit of the present invention. As used herein, the term “Limited of Detection” or “(LoD)” refers to the lowest quantity or concentration of an analyte in a sample that can be reliably detected. Tests conducted by the inventors support that the methods and/or detection kits of the present invention can achieve an overall sensitivity in the range of from about 85% to about 100%.

Specificity tests conducted by the inventors support that the methods and/or detection kits of the present invention are capable of detecting antigens or antibodies with specificity greater than about 95% to about 100%, or in the range of from about 99% to about 100%. Low false positive and low false negative rates can thus be achieved in the detection of antibodies or antigens in a sample using the methods and/or detection kits of the present invention.

Rapid detection of antibodies or antigens is possible using the methods of the present invention because of the simple steps in the methods, and/or the reduced number of steps. In some embodiments, the detection method of the present invention comprises a one-step procedure in preparing or processing the sample within the sample processing device 10. In some embodiments, detection results are obtained in less than two minutes beginning from the sample collection step to the result display step, or in some embodiments, less than one minute, or in some embodiments, in the range of from about seconds to about one minute.

### Example Method of Detecting Antigens

In one example embodiment, as illustrated in FIG. 6A, the test region 206 comprises a polymer 207 which is an antibody immobilized thereon. The antibody may be designed to capture target antigens such as viral antigens from the sample. In such an example embodiment, the polymer 44 conjugated to the nanoparticles 48 of the color conjugate 46 comprises an antibody designed to capture the target antigen present in the sample. In some embodiments, the passing of the mixture through the filter 40 causes target antigens (if a detectable amount is present in the sample) to bind to the polymer 44 (e.g., antibody in this example) of the color conjugate 46 to form a complex 209. The complex 209 may be released. The flowing of the mixture comprising the released complex 209 onto the test region 206 of the membrane 204 may cause the binding of the target antigen bound to the polymer 44 of the color conjugate 46 onto the polymer 207 (e.g., antibody in this example) immobilized on the test region 206. The presence of a color signal may be shown on the test region 206 if a detectable amount of target antigens are present in the sample. In the absence of a detectable amount of target antigens in the sample, complex 209 is not formed and the color conjugate 46 is released and is caused to flow onto the test region 206. Since no target antigens are present to bind to the polymer 44 conjugated to the nanoparticles 48 and thereby to the polymer 207 (e.g., antibody in this example) immobilized on the test region 206, no color signal will be displayed on the test region 206.

In some embodiments, as illustrated in FIG. 6B, the mixture comprising the released complex 209 and/or the color conjugate 46 is caused to flow onto the control region 208 of the membrane 204. The flowing of the mixture comprising the released complex 209 and/or the color conjugate 46 onto the control region 208 may occur before, after, or simultaneous to the flowing of the released complex onto the test region 206. The control region 208 may comprise a control polymer 213 such as a protein immobilized thereon. In some embodiments, the protein is Protein A or Protein A like molecules. In some embodiments, the flowing of the mixture comprising the released complex 209 and/or the color conjugate 46 onto the control region 208 causes the polymer 44 (e.g., antibody in this example) conjugated to the nanoparticles 48 of the color conjugate 46 to bind to the control polymer 213.

In some embodiments, the control polymer 213 is designed to bind to the polymer 44 (e.g., antibody) conjugated to the nanoparticles 48, thereby a color signal is expected to be displayed on the control region 208 if the test is performed properly. The absence of a color signal on the control region 208 is an indication that the test is not performed properly.

### Example Method of Detecting Antibodies

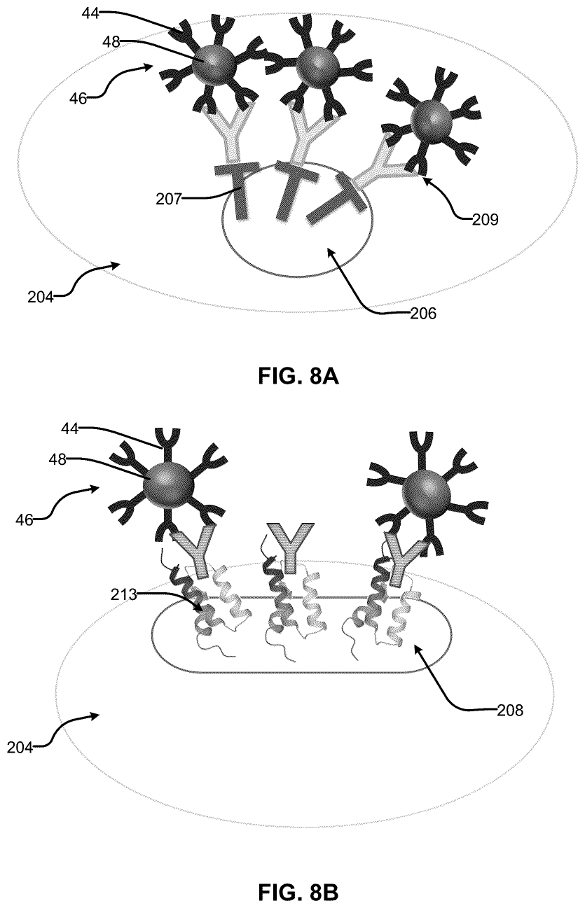

In another example embodiment, as illustrated in FIG. 8A, the test region 206 comprises a polymer 207 immobilized thereon. In these embodiments, the polymer 207 is a protein designed to capture an antibody from the sample. In such example embodiments, the polymer 44 conjugated to the nanoparticles 48 of the color conjugate 46 comprises a protein, such as Protein A or Protein-A like molecules, designed to bind to the target antibody present in the sample. In some embodiments, the passing of the mixture through the filter 40 causes target antibodies (if any detectable amounts are present in the sample) to bind to the polymer 44 of the color conjugate 46 to form the complex 209. The complex 209 may be released from the filter 40. The flowing of the mixture comprising the released complex 209 onto the test region 206 of the membrane 204 may cause the binding of the target antibody bound to the polymer 44 of the color conjugate 46 onto the polymer 207 (e.g., protein in this example) immobilized on the test region 206. The presence of a color signal may be shown on the test region 206 if detectable amounts of target antibodies are present in the sample. The absence of any detectable amounts of target antibodies in the sample will result in the lack of a color signal being displayed on the test region 206, since no target antibodies are present to bind to the polymer 44 conjugated to the nanoparticles 48 and thereby to the polymer 207 (e.g., antibody in this example) immobilized on the test region 206.

In some embodiments, the released complex 209 and/or the color conjugate 46 is caused to flow onto the control region 208 of the membrane 204. The flowing of the released complex 209 and/or the color conjugate 46 onto the control region 208 may occur before, after, or simultaneous to the flowing of the released complex 209 onto the test region 206. The control region 208 may comprise a control polymer 213 such as a protein immobilized thereon. In some embodiments, the protein is Protein A or Protein A like molecules. In some embodiments, the flowing of the mixture comprising the released complex 209 and/or the color conjugate 46 onto the control region 208 causes IgG antibodies that may be naturally present in the sample (such as in a blood sample) to bind to the control polymer 213. The polymer 44 (such as a protein) conjugated to the nanoparticles 48 of the color conjugate 46 may in turn bind to the IgG antibody. The control polymer 213 may be designed to bind to the polymer 44 (such as a protein) conjugated to the nanoparticles 48, thereby a color signal is expected to be displayed on the control region 208 if the test is performed properly. The absence of a color signal on the control region 208 is an indication that the test is not performed properly.

### EXAMPLES

### Example 1—Antigen Test

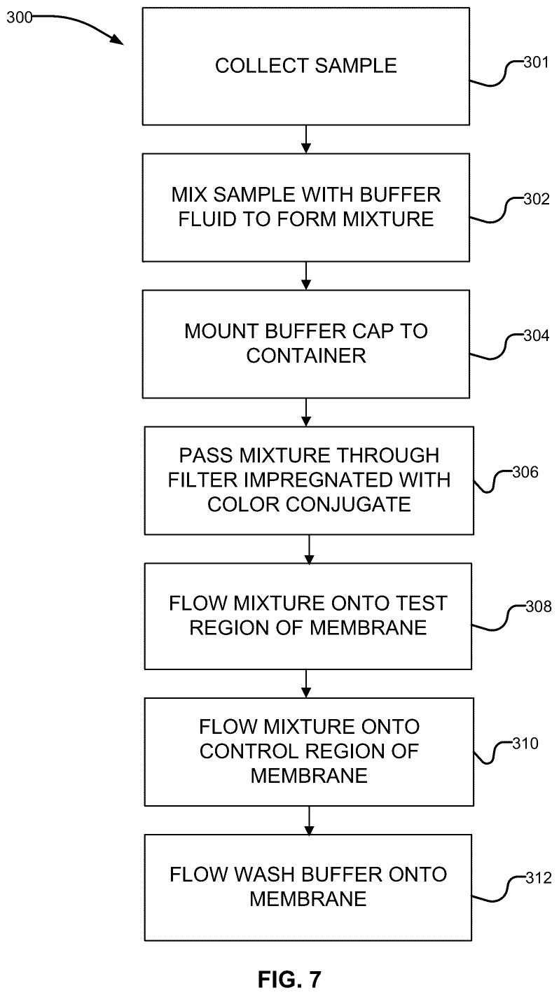

A detection kit of the type illustrated in FIGS. 4 and 5, and the process of performing the detection illustrated in FIGS. 6A, 6B, and 7 were used to detect severe acute respiratory syndrome coronavirus type 2 (SARS-CoV-2) from human samples collected from a nasal swab. In the sample embodiment, the filter 40 is coated with the color conjugate 46 comprising colloidal gold nanoparticles and a monoclonal antibody (Au-mAb) specific for the detection of SARS-CoV-2. Once dried, the filter 40 coated with the color conjugate 46 was snuggly fitted within the buffer cap 18 of the sample processing device 10. The test device 202 comprises a nitrocellulose membrane at the test region 206 and the control region 208. Anti-human IgG and IgM antibodies specific for the detection of the monoclonal antibody specific for the detection of SARS-CoV-2 were immobilized on the test region 206. A Protein A molecule was immobilized on the control region 208. The buffer container 12 was filled with the buffer fluid 14 comprising a sample extraction buffer. A fluid sample was added to the buffer container 12, and mixed with the sample extraction buffer to form a mixture. The buffer cap 18 was mounted onto the buffer container 12 to close the buffer container 12. The mixture was caused to pass through the filter 40 to flow onto the test region 206 and the control region 208. A color indicator, indicating the results, may be displayed on the control region 208 and the test region 206 (if a detectable amount of SARS-CoV-2 antigens is present in the sample).

### Example 2—Antibody Test

A detection kit of the type illustrated in FIGS. 4 and 5, and the process of performing the detection illustrated in FIGS. 7, 8A and 8B were used to detect antibodies against severe acute respiratory syndrome coronavirus type 2 (SARS-CoV-2) from human serum and whole blood samples. In the sample embodiment, the filter 40 is coated with the color conjugate 46 comprising colloidal gold nanoparticles and Protein A (Au-PA). Once dried, the filter 40 coated with the color conjugate 46 was snuggly fitted within the buffer cap 18 of the sample processing device 10. The test device 202 comprises a nitrocellulose membrane at the test region 206 and the control region 208. SARS-CoV-2 spike proteins (i.e., spike proteins exposed on the surface of the SARS-CoV-2 virus) and nucleocapsid protein (a protein located within the SARS-CoV-2 virus capsid) were immobilized on the test region 206. A Protein A molecule was immobilized on the control region 208. The buffer container 12 was filled with the buffer fluid 14 comprising a sample extraction buffer. A sample (serum or whole blood samples) was added to the buffer container 12, and mixed with the sample extraction buffer to form a mixture. The buffer cap 18 was mounted onto the buffer container 12 to close the buffer container 12. The mixture was caused to pass through the filter 40 to flow onto the test region 206 and the control region 208. A color indicator, indicating the results, may be displayed on the control region 208 and the test region 206 (if a detectable amount of antibodies against SARS-CoV-2 is present in the sample).

Specificity tests were conducted on the Examples 1 and 2 detection kit and processes. The test results support that the methods of the present invention show no cross-reaction with other commonly found pathogens including Middle East respiratory syndrome coronavirus (MERS-CoV), Influenza A virus subtype H1N1, Human Parainfluenza Viruses (HPIVs), Respiratory syncytial virus (RSV), Rhinovirus, Adenovirus, Enterovirus, Enterovirus group D, Epstein-Barr virus (EBV), Human cytomegalovirus (CMV), Rotavirus, Varicella-zoster virus (VZV), Human Metapneumovirus (HMPV), Mycoplasma pneumoniae, Chlamydia pneumoniae, Haemophilus influenza (Hi), Legionella, Mycobacterium tuberculosis (M. tb), Group A Streptococcus or Streptococcus pyogenes, Streptococcus pneumoniae, Staphylococcus epidermidis, Staphylococcus aureus, Pneumocystis pneumonia (PCP), and Candida albicans.

The inventors have tested the following potentially interfering substances: Mucin, Whole Blood, Beclomethasone, Dexamethasone, Flunisolide, Triamcinolone acetonide, Budesonide, Mometasone, Fluticasone, Phenylephrine, Oxymetazoline, Menthol, Benzocaine, Zicam, Sore Throat Phenol Spray, alpha interferon, Zanamivir, Ribavirin, Oseltamivir, Peramivir, Lopinavir, Ritonavir, Abidor, Levofloxacin, Azithromycin, Ceftriaxone, Meropenem, Mupirocin, Tobramycin, Histamine Dihydrochloride, and Biotin. Results have shown that the test concentrations of these potentially interfering substances were at levels which have no impact on the Examples 1 and 2 detection kit and processes.

Throughout the foregoing description and the drawings, in which corresponding and like parts are identified by the same reference characters, specific details have been set forth in order to provide a more thorough understanding to persons skilled in the art. However, well known elements may not have been shown or described in detail or at all to avoid unnecessarily obscuring the disclosure.

As will be apparent to those skilled in the art in the light of the foregoing disclosure, many alterations and modifications are possible in the practice of this invention without departing from the scope thereof. Accordingly, the description and drawings are to be regarded in an illustrative, rather than a restrictive, sense.

1. A sample processing device (10) for the detection of an antibody or antigen in a sample comprising:
a buffer container (12) for receiving a buffer fluid (14) therein;
a buffer cap (18) snuggly mountable to an open end (16) of the buffer container, the buffer cap having a fluid outlet end (29) and an opposing fluid inlet end (22), the fluid inlet end being contactable with the buffer container; and
a filter (40) arranged at the fluid inlet end of the buffer cap, the filter being coated with a color conjugate (46) comprising a color reagent (42) coupled to a polymer (44), wherein the filter and the color conjugate form a single unit.

2. The sample processing device according to claim 1, wherein the color conjugate comprises colloidal nanoparticles (48).

3. The sample processing device according to claim 1, wherein the color conjugate comprises a colloidal conjugate solution comprising colloidal nanoparticles and the polymer.

4. The sample processing device according to claim 3, wherein the colloidal nanoparticles comprises colloidal gold nanoparticles.

5. The sample processing device according to claim 4, wherein the colloidal gold nanoparticles are produced from a solution comprising hydrogen tetrachloroaurate (HAuCl₄) and/or gold chloride (AuCl₃).

6. The sample processing device according to claim 5, wherein a weight concentration of the hydrogen tetrachloroaurate and/or gold chloride in the solution is greater than about 0.01%.

7. The sample processing device according to claim 5, wherein a weight concentration of the hydrogen tetrachloroaurate and/or gold chloride in the solution is greater than about 0.05%.

8. The sample processing device according to claim 5, wherein a weight concentration of the hydrogen tetrachloroaurate and/or gold chloride in the solution is greater than about 0.1%.

9. The sample processing device according to claim 5, wherein a weight concentration of the hydrogen tetrachloroaurate and/or gold chloride in the solution is in the range of from about 0.1 to about 1.0%.

10. The sample processing device according to claim 5, wherein a weight concentration of the hydrogen tetrachloroaurate and/or gold chloride in the solution is in the range of from about 0.1 to about 0.5%.

11. The sample processing device according to claim 5, wherein the solution further comprises one or both of a reducing agent and a stabilizer.

12. The sample processing device according to claim 5, wherein the solution is heated to a temperature in the range of from about 20° C. to about 100° C. to produce the colloidal gold nanoparticles.

13. The sample processing device according to claim 5, wherein the colloidal conjugate solution has an optical density (OD) at about 500 nm to about 550 nm wavelength in the range of from about 10 to about 30.

14. The sample processing device according to claim 1, wherein the polymer comprises an antibody or antigen for the detection of the antibody or antigen in the sample.

15. The sample processing device according to claim 1, wherein the fluid inlet end of the buffer cap comprises a base (24), a wall (26) projecting outwardly from the base, and an opening (28) defined by the wall.

16. The sample processing device according to claim 15, wherein the filter is snuggly fitted within the opening.

17. A kit for detection of antibodies or antigens in a sample comprising:
a test device comprising a membrane having a test region and a control region;
a sample processing device according to claim 1; and
a buffer.

18. The kit according to claim 17, wherein the control region of the membrane comprises a polymer immobilized thereon arranged to bind to the polymer coupled to the color reagent.

19. The kit according to claim 17, wherein the control region of the membrane comprises a polymer immobilized thereon arranged to bind to an IgG antibody in the sample.

20. The kit according to claim 17, wherein the test region of the membrane comprises a polymer immobilized thereon arranged to bind to a target antibody or antigen in the sample.
